(19) United States
(12) Patent Application Publication
(10) Pub. No.: US 2006/0025742 A1
(43) Pub. Date: Feb. 2, 2006

(54) Absorbent article with color surfaces

(75) Inventors: Margaret Henderson Hasse, Wyoming, OH (US); Charles John Berg JR., Wyoming, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/902,820

(22) Filed: Jul. 30, 2004

(51) Int. Cl. A61F 13/20

(52) U.S. Cl. 604/385.18; 604/385.01

(57) ABSTRACT

A tampon having an absorbent member. Optionally, the tampon can have an overwrap and/or a skirt portion. The absorbent member and the overwrap have an imparted color in which each of the imparted colors for the aforementioned elements have an L* value of greater than about 60 and a C* value of greater than about 6.

### FIELD OF THE INVENTION

This invention is directed to disposable absorbent articles, such as diapers, training pants, adult incontinence articles, feminine protection articles and the like wherein the article comprises elements that have a specified color according to International Commission on Illumination L*a*b* system (hereinafter “CIELab” from the French title Commission Internationale de l'Eclairage).

### BACKGROUND OF THE INVENTION

Absorbent articles such as disposable diapers, pull-on diapers, training pants, tampons, pantiliners, incontinence briefs, and the like, are well known in the art and are highly effective for absorbing and containing urine and other bodily exudates. Since their introduction into the marketplace, absorbent articles have continued to improve regarding fit and functionality. For example, tampons are constructed to provide a better fit with the wearer of the article and are able to contain large quantities of bodily exudates without leakage or adverse impact to the wearer's skin.

Comparatively little effort has been expended on making a tampon that is attractive before and after insertion into the vaginal cavity. Accordingly, feminine protection articles have not advanced quite as significantly in regard to aesthetic design. Since the inception of commercially available feminine protection articles, the feminine protection articles' predominant color is that which is inherent within the materials selected for construction. The materials are generally a white color often achieved by the use of pigments such as titanium dioxide or by bleaching. The white color has historically been common to absorbent articles, in general including diapers, adult incontinence products, underarm sweat products, collar inserts, tampons and pantiliners to communicate a hygienic condition.

To provide the appealing aesthetic design of a colored tampon, elements of the feminine protection article must be matched. Ensuring that colors within a feminine protection article match is important but difficult to achieve. A feminine protection article having matching colors communicates to the consumer that the product is of high quality and differentiates the product from competitors' products. A feminine protection article having matching colors is holistically pleasing and is believed to be preferred by consumers. However, absorbent articles are difficult products to color match. Feminine protection articles are comprised of a variety of materials. Printing, coating, or impregnating are well known methods for imparting color, but not all materials used in feminine protection article construction are amenable to each of these methods. As a result, one material may only be colored by printing or coating while a different material adjacent to the first may only accept impregnation of color. Even if two different materials are amenable to a single method for imparting color, the imparted color may appear mismatched between the two materials in the finished product. Many surfaces of a feminine protection article are laminates whereby the color-bearing layer is covered by a second layer. The resulting laminate exhibits a muted color compared to the color of the color-bearing layer. Furthermore, the texture of the material may also adversely impact the color imparted.

A further problem in color matching is determining when two surfaces have matching colors. Reliance on human discernment of color differences or matching is undependable because color differentiation is a highly variable personal characteristic. As a result, what appears to be color matched to one observer may be distinguishable to another. Furthermore, quantifying the highly qualitative concept of human discernment of color differences or matching between two or more surfaces is very difficult.

### SUMMARY OF THE INVENTION

In response to the difficulties and problems described herebefore, a disposable absorbent article is disclosed having color matched elements according to CIELab color space calculations. Specifically, a disposable absorbent article is disclosed comprising an absorbent comprising at least one externally visible surface comprising an imparted color. In one embodiment, a tampon comprises an externally visible surface comprising an imparted color. The imparted color of the tampon has an L* value of greater than about 60 and a C* value of greater than about 6. In another embodiment, a tampon comprises an absorbent material. The absorbent material has a first surface opposed to a second surface. The absorbent material comprises an externally visible surface comprising an imparted color. The imparted color has an L* value of greater than about 60 and a C* value of greater than about 6. Optionally, the absorbent article could comprise an overwrap comprising an externally visible surface comprising an imparted color. For example, the imparted color of the absorbent material and the imparted color of the overwrap independently have an L* value of greater than about 60 and a C* value of greater than about 6.

### BRIEF DESCRIPTION OF THE DRAWINGS

While the specification concludes with claims particularly pointing out and distinctly claiming the subject matter that is regarded as forming the present invention, it is believed that the invention will be better understood from the following description taken in conjunction with the accompanying drawings, in which:

FIG. 1 is a perspective view of a tampon of the present invention incorporating a compressed absorbent member and an overwrap covering the exterior surface and forming a skirt.

FIG. 2 is a plan view of an assembled absorbent material and overwrap prior to compression.

FIG. 3 is a longitudinal cross section of the absorbent material and overwrap prior to compression.

FIG. 4 is an illustration of the Commission Internationale de l'Eclairage L*a*b* color space.

### DETAILED DESCRIPTION OF THE INVENTION

The present invention comprises a tampon with an externally visible surface. The externally visible surface has an imparted color. The imparted color has an L* value of greater than about 60 and a C* value of greater than about 6. Another aspect of the present invention matches the imparted colors of at least two elements of the tampon. Namely, the two elements can be the overwrap which covers at least a portion of the exterior surface of the compressed absorbent member and the compressed absorbent member. Optionally, the tampon could have a skirt which extends beyond the withdrawal end. The skirt, constructed from the overwrap, draws bypassed fluid from the bottom of the vagina, thereby increasing absorbency and minimizing bypass discharge.

“Absorbent articles” as referred to herein are primarily sanitary napkins, pantiliners, or incontinence pads or any other type of article that can be worn in the crotch region of an undergarment. However, articles such as sweat-absorbent underarm pads, nursing pads or collar inserts can also benefit from the present invention. Baby diapers, adult incontinence diapers, and human waste management devices may benefit from the present invention even though they are conventionally not worn in conjunction with an undergarment.

“Color” as referred to herein includes any color, i.e., white, black, red, blue, violet, orange, yellow, green, and indigo as well as any declination thereof or mixture thereof.

As used herein, the term “element” refers to the separate and discrete parts that must be united together to form the tampon. Each element may comprise one or more lamina. Furthermore, elements may share a common, continuous lamina that has not been subjected to a coloration technique yielding an imparted color. However, elements may not share a common, continuous lamina that has been subjected to a coloration technique yielding an imparted color. The main elements of the tampon can be the absorbent material, the overwrap, and/or the skirt.

The term “disposable” is used herein to describe absorbent articles that are not intended to be laundered or otherwise restored or reused as absorbent articles (i.e., they are intended to be discarded after a single use and, preferably to be recycled, composted or otherwise disposed of in an environmentally compatible manner).

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, the term “externally visible surface,” refers to a visible surface on either side of the absorbent article that a human viewer may visually discern with the unaided eye (excepting standard corrective lenses adapted to compensate for near-sightedness, farsightedness, or astigmatism) in standard lighting conditions from a point of reference of viewing the article before proper application. The externally visible surface (i) is visually discernible without tearing, ripping, puncturing, or otherwise mutilating or damaging the article and (ii) has been subjected to a coloration technique resulting in an imparted color. Preferably, the externally visible surface of an element may be color matched according to the present invention.

As used herein, the term “inner region,” refers to an externally visible surface that a human viewer may not visually discern with the unaided eye (excepting standard corrective lenses adapted to compensate for near-sightedness, farsightedness, or astigmatism) in standard lighting conditions from a point of reference of viewing the article in plan view (i.e., the article is stretched such that the inner region is substantially planar).

As used herein, the term “imparted colors” are those colors printed, coated, or impregnated onto or into the externally visible surface of the tampon. Imparted colors are those colors with a CIELab C* (formula provided below) of greater than about 6.

As used herein, “standard lighting conditions” refer to lighting conditions in which human vision operates efficiently (e.g., the human eye is able to discern complex patterns, shading, and colors). Specifically, for the purposes of describing this invention, standard lighting conditions are at least one of the following:
a) natural illumination as experienced outdoors during daylight hours,
b) the illumination of a standard new (i.e. not used) 100 watt incandescent white light bulb at a distance of 2 meters from the object to be illuminated with no intervening articles (e.g. lamp shade), or
c) as defined by CIE D65 standard illuminant lighting at 800 lux to a 1964 10 degree CIE standard observer.

As used herein, the term “color matched” refers to colors that fall within a prescribed color space volume, that have a prescribed color space hue difference, and/or that have a prescribed total color difference.

As used herein the term “tampon” refers to any type of absorbent structure that is inserted into the vaginal canal for the absorption of fluid therefrom. Typically, tampons are constructed from an absorbent material that has been compressed into a vaginally insertable shape.

As used herein the terms “pledget” or “tampon pledget” are intended to be interchangeable and refer to a construction of absorbent material prior to the compression of such construction into a tampon.

As used herein, the terms “vaginal cavity” and “within the vagina” refer to the internal genitalia of the human female in the pudendal region of the body.

The term “folded” as used herein, is the configuration of the compressed absorbent member that may be incidental to radial and/or lateral compression of the absorbent material or may purposely occur prior to a compression step. Such a configuration is readily recognizable, for example, when the absorbent material abruptly changes direction such that one part of the absorbent material bends and lies over another part of the absorbent material. When overwrap is placed on the absorbent material prior to compression, it too may be “folded.”

The term “rolled” as used herein, is the configuration of the compressed absorbent member after winding the absorbent material with or without the overwrap or overwraps in a spiral round (i.e. the material is round upon itself).

As used herein the term “skirt” or “skirt portion” refers to the portion of the overwrap that extends beyond the withdrawal end of the absorbent material or compressed absorbent.

As used herein “overwrap” refers to the liquid pervious material substantially covering the exterior surface of the compressed absorbent member and optionally extending below the withdrawal end to form a skirt portion. The overwrap may comprise a fibrous non-woven material comprising a blend of synthetic and natural fibers. Suitable synthetic fibers and natural fibers and their ratios are described in Ser. No. 09/993,988, filed Nov. 16, 2001, entitled “Tampon With Fluid Wicking Overwrap with Skirt”, to “Hasse, et al.” There are number of techniques in which a blend of fibers form overwrap 40. Suitable techniques are described in Ser. No. 09/993,988, filed Nov. 16, 2001, entitled “Tampon With Fluid Wicking Overwrap with Skirt”, to “Hasse, et al.”

The overwrap 40 may possess a horizontal fluid wicking capacity of at least about 2, alternatively from about 3 to about 6 grams of fluid per gram of tampon at a 500 second interval. In one embodiment, the overwrap is 50% rayon, 50% polyester hydroentangled. Another embodiment includes a material that is dual layered with an outside and inside layer, made in accordance with U.S. Pat. No. 5,273,596. In this case, the outside layer is a 75% hydrophilically treated polypropylene with a 2.2 dpf and 25% 1.5 dpf rayon. The inside layer is 25% hydrophilically treated polypropylene with a 2.2 dpf and 75% 1.5 dpf rayon. The basis weights of the layers can vary, having from about 10 to about 15 grams per square meter in each layer. The resultant material is a 50% rayon 50% polypropylene thermally bonded blend with a basis weight from about 20 to about 30 grams per square meter. Both materials are produced by BBA Corporation of South Carolina, U.S.A.

As used herein “compressed” refers to pressing or squeezing together or otherwise manipulating the size, shape, and/or volume to obtain a tampon having vaginally insertable shape.

Unless specifically stated otherwise, as used herein a first material is “substantially covering” or “substantially covers” a second material when the first material covers at least about 75%, typically at least about 90% of the surface area of the second material.

The term “joined” or “attached” as used herein, encompasses configurations in which a first element is directly secured to second element by affixing the first element directly to the second element; configurations in which the first element is indirectly secured to the second element by affixing the first element to intermediate member(s) which in turn are affixed to the second element; and configurations in which first element is integral with second element; i.e., first element is essentially part of the second element.

As used herein, “cm” is centimeter, “mm” is millimeters, “ml” is milliliters “g” is grams, “gsm” is grams per meter squared, “dpf” denier per fiber, “sec” is seconds.

While particular embodiments of the present invention have been illustrated and described, it would be obvious to those skilled in the art that various other changes and modifications can be made without departing from the spirit and scope of the invention.

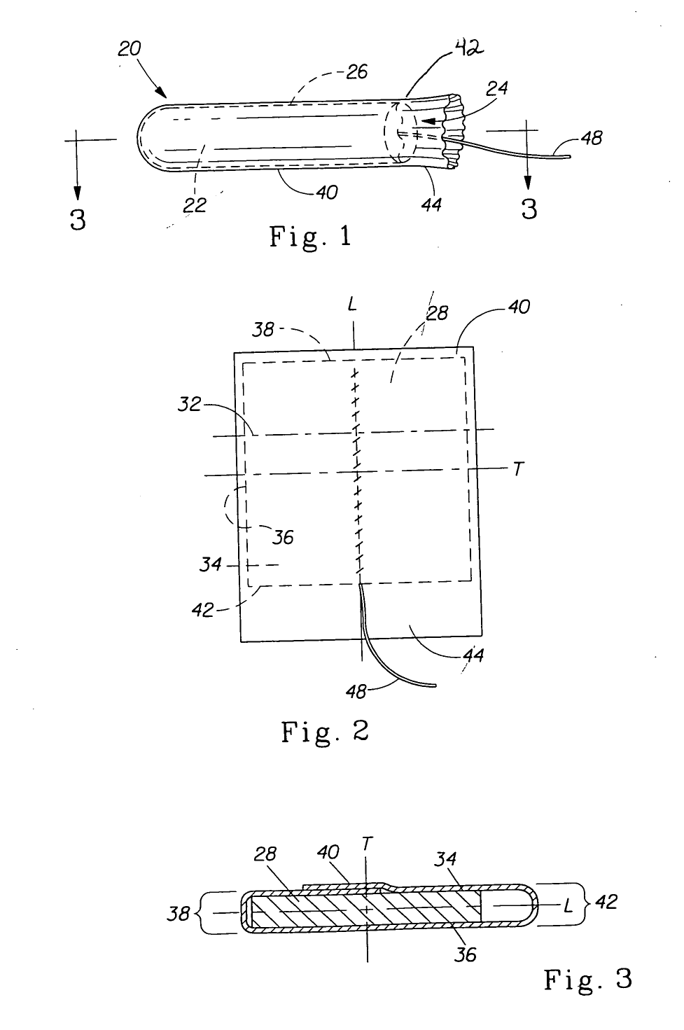

FIG. 1 shows in partial cut away perspective view of absorbent tampon 20. In general, tampon 20 comprises a compressed absorbent member 22 and a fluid permeable overwrap 40 that covers absorbent member 22. The compressed absorbent member 22 has an exterior surface 26. The compressed absorbent member has an inner region 24 shown in greater detail in FIG. 2 below.

Overwrap 40 may extend beyond one end of absorbent member 22 to form a skirt portion 44. Tampons 20, including overwraps 40 for use as the body contacting surface thereof, are well known in the art and need no detailed description of various alternative and optional designs. The present invention, however, is not limited to a structure having the particular configuration shown in the drawings.

The exterior surface 26 of the compressed absorbent member 22 is substantially covered by the overwrap 40. This compressed primary absorbent also includes a skirt portion 44. The skirt portion 44 is comprised of overwrap 40 extending over the withdrawal end 42 of the absorbent material 28 as shown in greater detail in FIG. 2. A removal means, such as string 48 can be provided to facilitate removal of the tampon 20 after use.

FIG. 2 shows the construction of absorbent material 28 prior to the compression of such construction into the tampon 20 (FIG. 1). To form a tampon 20 (FIG. 1) ready for use, the absorbent material 28 and the overwrap 40 are typically compressed and optionally heat conditioned in any suitable conventional manner. FIG. 2 shows the overwrap 40 substantially covering the absorbent material 28 prior to compression. The absorbent material 28 has a first surface 34 opposed to the second surface 36 and an insertion end 38 opposed to a withdrawal end 42. The absorbent material 28 has both a longitudinal axis and a transverse axis indicated by the lines marked “L” and “T” respectively.

The first surface 34 and opposed second surface 36 of the absorbent material 28 is substantially covered with overwrap 40. The overwrap 40 is positioned around the absorbent material 28 so that the overwrap 40 may be proximate with the insertion end 38. The overwrap 40 can overlap at the seam 32. The overwrap 40 may extend beyond the withdrawal end 42 to form a skirt portion 44. In one embodiment, the tampon 20 includes a withdrawal means 48.

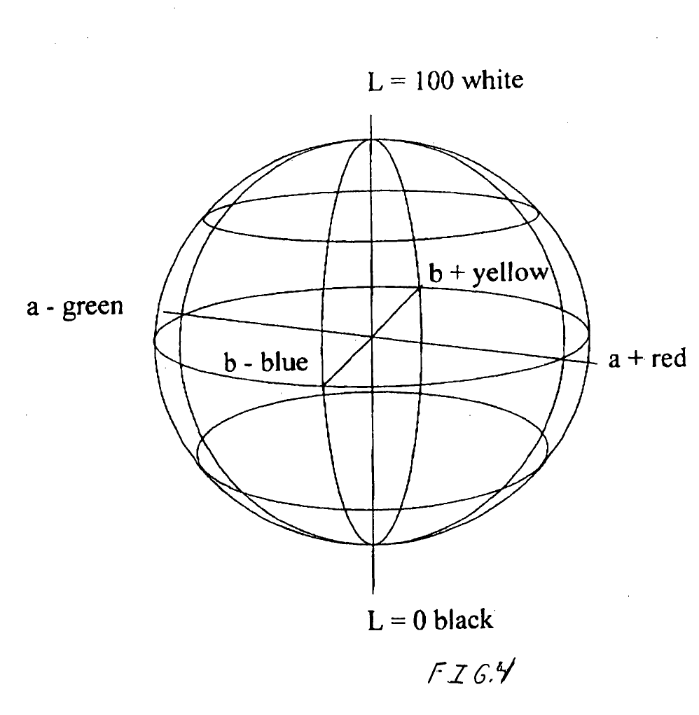

FIG. 3 shows a longitudinal cross section of the absorbent material 28 and overwrap 40 prior to compression. The absorbent material 28 has a first surface 34 opposed to the second surface 36 and an insertion end 38 opposed to a withdrawal end 42. The absorbent material 28 is located in the center of the longitudinal cross-section. The overwrap 40 is positioned around the first surface 34 of the absorbent material 28 and opposed to the second surface 36.

Referring to FIG. 1, a tampon 20 of the present invention is generally constructed from a variety of elements that are joined together. The various elements of tampon construction generally have at least one externally visible surface. It is desirable for the tampon 20 to comprise an absorbent member 22 and an overwrap 40. A surface initially not an externally visible surface may subsequently become an externally visible surface during storage, application, wear, or disposal. Generally, the elements of the tampon 20 will contain two surfaces typically oriented with inner region 24 (FIG. 1), which is typically an internal surface and an externally visible surface. Color matching may occur between interior surfaces, externally visible surfaces, or both.

According to the present invention, when the tampon has at least two elements of the tampon with an L* value of greater than about 60 and a C* value of greater than about 6, the colors have a CIELab value of volume of less than 190. Typically this will require only moderate modification of the material composition while maintaining the majority of the conventional material characteristics. In the following, examples of materials, which are particularly beneficial for the use in the absorbent articles according to the present invention, are mentioned. Those skilled in the art will readily be able to identify alternative materials, which can also be used in the context of the absorbent articles according to the present invention.

The CIELab value, utilized herein to define the darkness/lightness of the materials of the absorbent articles according to the present invention, are units of color measurement in the aforementioned CIELab system. The absorbent articles herein, and hence the materials of which the absorbent articles are made of have an L* value of greater than about 60 and a C* value of greater than about 6 color.

The various colors exhibited by the tampon 20 may generally be divided into two categories. Inherent colors are typically colors with little to no chroma. Qualitatively, inherent colors represent the white to off-white colors traditionally associated with tampons. The inherent color is usually the color of the raw material that comprises the externally visible surfaces. The whiteness of the material may be manipulated, such as by bleaching, printing, coating, or impregnating a substrate with titanium dioxide pigment. More specifically and quantitatively, inherent colors are those colors with a CIELab chroma value, C*, of less than about 6 (formula provided below). Imparted colors are those colors printed, coated, or impregnated onto or into the externally visible surface of the tampon. Imparted colors are those colors with a CIELab C* of greater than about 6. Externally visible surfaces generally contain an inherent color and may contain one or more imparted colors.

It is further recognized that an externally visible surface having an imparted color may comprise a single stratum or may be a laminate comprising more than one lamina. Regarding a single stratum, the imparted color is imparted onto or within that stratum. Regarding a laminate, color may be imparted on one or more of the lamina. For example, a laminate generally may comprise an exterior lamina (i.e., the layer closest in proximity within the line of sight of an observer) and at least one interior lamina. One or more of the lamina may have an imparted color. For example, the laminate may comprise an exterior lamina with no imparted color and at least one interior lamina with an imparted color. When the tampon 20 is viewed, the imparted color of the interior lamina provides the coloration for the laminate as a whole. Thus, the perceptible color of the laminate as a whole is the result of the exterior lamina and other optional interior laminae possibly diffusing and shifting the imparted color of the interior lamina. As a result, the imparted color of the laminate as a whole may be different (i.e., not color matched) as compared to the imparted color of the individual lamina if viewed in isolation. Additionally, the exterior lamina and one or more interior laminae may exhibit imparted colors such that the imparted color of the individual lamina when viewed in isolation are not color matched with the imparted color of the laminate as a whole.

The imparted color on an externally visible surface may be provided by a variety of coloration techniques that are well known in the art. Color may be provided to an externally visible surface by coloration techniques including, but not limited to, printing, coating, and impregnating. Various printing methods may be used to impart color including, but not limited to, letterpress, flexography, gravure, offset lithography, screen, and inkjet. All methods are well known in the art.

Letterpress, the oldest method of printing, involves ink or other equivalent material being applied to the top of a raised surface. This surface is pressed against a substrate, thus transferring the ink to the substrate. Flexographic printing uses a printing plate, often cylindrical, made of rubber, plastic, or other flexible material. Ink is applied to a raised image on the plate. The plate is then placed in contact with a substrate, and ink is transferred to the substrate. Water-based and solvent-based inks are used in flexography. Most inks used are fast drying which makes flexography particularly well-suited for printing on plastics, foils, compressible surfaces, and other nonabsorbent substrate.

Gravure printing uses a print cylinder having depressions of varying depths that are etched into the cylinder. This method of printing is performed by partially immersing the etched cylinder (generally about a fourth of the cylinder diameter) into an enclosed fountain or trough of ink. The etched cells, which produce the image, are filled with ink, and the surface of the cylinder also becomes coated with ink. Since the surface of the cylinder is non-image producing, ink is not desirable on the cylinder surface. This undesired ink is removed by a doctor blade or knife which wipes all of the surface ink from the cylinder. As the printing cylinder comes in contact with the substrate, the ink contained within the cells is transferred to the substrate. Gravure is ideal for continuous printing operations and the printing of very long runs. Generally, solvent-based inks are used in gravure printing.

Lithographic printing, or offset lithography, is a printing method that utilizes surface characteristics on an image carrying offset plate. Offset plates are typically made from a thin paper, plastic, or a metal sheet, which once exposed and processed can be wrapped around a cylinder of a press for printing. The offset plate contains two areas: an image area that is hydrophobic and a non-image area that is hydrophilic. While the basic principle is common, there are many differences between offset plates and the method they use to separate the image from the non-image areas. Generally, ink adheres to the hydrophobic image area while being repelled from the hydrophilic non-image area. The ink and watered offset plate may be printed on a second cylinder usually coated in rubber. The second cylinder then off-sets this ink and water impression onto the substrate.

Screen printing utilizes a porous screen made from silk or other polymeric material. The screen is attached to a frame. A stencil is produced on the screen either photo-mechanically or manually. The non-printing areas are protected by the stencil. Printing is done on the substrate under the screen by applying a viscous ink to the screen. The ink is forced through the fine openings of the screen with a rubber squeegee or roller.

Inkjet printing is a non-impact dot-matrix technology where ink droplets are jetted from a small aperture directly to specified positions on a medium to create an image. Inkjet printing may be done on a continuous method or a drop-on-demand method. Continuous inkjet printing involves a continuous stream of ink droplets. Generally, the ink droplets may be charged by a charge electrode. If the droplets are not charged, the droplet travels directly to the substrate through and unimpeded by a voltage carrying plate. Droplets that are charged are deflected by the voltage carrying plate. If diverted, the droplet is captured and recirculated prior to reaching the substrate. Another continuous inkjet method charges all droplets and the voltage plate controls droplet placement onto the substrate or diversion. Drop-on-demand inkjet printing, as the name implies, provides an ink droplet only when needed. Droplets are formed by a variety of methods with thermal and piezoelectric drop formation being most common. Thermal inkjet printing involves the ink droplets being expelled from a nozzle by the rapid expansion of an ink vapor bubble created by a small heater. Piezoelectric inkjet printing involves the ink droplets being expelled from a nozzle by a pressure wave created from the expansion of a piezoelectric ceramic upon application of a voltage. Inkjet printing techniques are well known in the art as described in Hue. P. Le, Progress and Trends in Ink-Jet Printing Technology, Journal of Imagining Science and Technology, Vol. 42, pages 49-62.

A variety of coating techniques may be used to impart color onto an externally visible surface. Suitable coating techniques are well-known in the art and include, but are not limited to, bead extruders, slot die coaters, spray nozzles, dip tanks, brushes, and combinations thereof. Suitable slot die coaters include the EP11 Applicator available from Nordson Corp., Dawsonville, Ga. or the MR1300 Slot Die Coater available from ITW Dynatec Americas, Hendersonville, Tenn. Suitable coatings include, but are not limited to, adhesives, varnishes, latexes, lotions, waxes, and paraffins. The coatings generally will contain a dye, pigment, or combination.

Color may be imparted to an externally visible surface by way of impregnation of a colorant into a substrate or by limited coating onto a substrate surface. Colorants such as dyes, pigments, or combinations may be impregnated in the formation of substrates such as polymerics, resins, or nonwovens. For example, the colorant may be added to molten batch of polymer during film, fiber, or filament formation. In another non-limiting example, the colorant may be added to the polymer on the visible exterior surfaces such as a co-extruded or coat extruded material such as sheath-core fibers, multilayer films, etc.

Referring to FIG. 2, the imparted color is matched according to the Commission Internationale de l'Eclairage L*a*b* color space (hereinafter “CIELab”). CIELab is a mathematical tristimulus color scale based on the CIE 1976 standard. CIELab allows colors to be described quantitatively and with precision. As presented in FIG. 1, CIELab allows a color to be plotted in three-dimensional space analogous to the Cartesian xyz space. CIELab has the colors green to red on what is traditionally the x-axis in Cartesian xyz space. CIELab identifies this axis as the a-axis. A negative a* value represents green and a positive a* value represents red. CIELab has the colors blue to yellow on what is traditionally the y-axis in Cartesian xyz space. CIELab identifies this axis as the b-axis. Negative b* values represent blue and positive b* values represent yellow. CIELab has lightness on what is traditionally the z-axis in Cartesian xyz space. CIELab identifies this axis as the L-axis. The L*-axis ranges in value from 100, which is white, to 0, which is black. An L* value of 50 represents a mid-tone gray (provided that a* and b* are 0). Any color may be plotted in CIELab according to the three values (L*, a*, b*).

The three-dimensional CIELab allows the three color components of chroma, hue, and lightness to be calculated. Within the two-dimensional space formed from the a-axis and b-axis, the components of hue and chroma can be determined. Chroma is the relative saturation of the perceived color and is determined by the distance from the origin as measured in the a*b* plane. Chroma, for a particular (a*, b*) set is calculated according to the following formula: C*=\sqrt{a*²+b*²}. For example, a color with a*b* values of (10,0) would exhibit a lesser chroma than a color with a*b* values of (20,0). The latter color would qualatively be perceived as being more red than the former. Hue is the relative red, yellow, green, and blue in a particular color. A ray can be created from the origin to any color within the two-dimensional a*b* space. Hue is the angle measured from 0° (the positive a*-axis) to the created ray. Hue can be any value of between 0° to 360°. Lightness is determined from the L* value with higher values being more white and lower values being more black.

The testing and comparison of externally visible surfaces occurs between discrete elements of tampon 20 construction each having at least one externally visible surface. A permissible comparison would be, for example, between the externally visible surface of the overwrap 40 and the externally visible surface of the absorbent member 22. A comparison should not be done between like elements. For example, a comparison should not be done between two sample regions on a single element of tampon 20 construction (e.g., a comparison between two points on an overwrap). Externally visible surfaces were tested for reflective color utilizing the following standardized procedure.

Ideally, after choosing two elements which have an imparted color comprising an L* value of greater than about 60 and a C* value of greater than about 6, the imparted colors for at least two of the elements of the tampon 20 will occupy a CIELab color space volume of less than about 190. Again, the elements of the tampon 20 are the absorbent member 22 and the overwrap 40. Each element has an imparted color. The externally visible surfaces are analyzed according to the Test Method described below. Upon analysis, the inherent color of an element comprising an externally visible surface will yield L*, a*, and b* coordinates. The CIELab color space volume is then calculated utilizing the formula presented above. The resulting volume will preferably be less than about 190. More preferably, the resulting volume will be less than about 150. Even more preferably, the resulting volume will be less than about 100.

Color matching of two or more elements comprising an externally visible surface, wherein each externally visible surface comprises an imparted color, can be determined by color space volume, total color difference, and/or hue difference.

i. Color Space Volume

One embodiment of the present invention is color matching of two or more externally visible surfaces of differing elements such that the imparted colors will have a defined CIELab color space volume. In other words, the color space volume represents how matchable one or more colors are to one another. The match is defined by the boundary surface and depends on the position of the color in the color space. Characterizing color matching within a volume is desirable such that the volume accounts for and considers all three dimensions within CIELab. While not being limited to the theory, such a three-dimensional measurement is believed to more fully characterize the difference in two colors. The CIELab color space volume (V), for a first color (L*₁, a*₁, b*₁) and a second color (L*₂, a*₂, b*₂), is calculated according to the following formula:

$$V = \frac{4}{3} π \vert \frac{Δ {L}^{*}}{2} \vert \vert \frac{Δ {a}^{*}}{2} \vert \vert \frac{Δ {b}^{*}}{2} \vert .$$

Within the formula, ΔL* is the difference in L* values between the two colors and is calculated by: ΔL*=L*₂−L*₁. The Δa* is the difference in a* values between the two colors and is calculated by: Δa*=a*₂−a*₁. The Δb* is the difference in b* values between the two colors and is calculated by: Δb*=b*₂−b*₁. The CIELab color space volume results in a solid substantially ellipsoidal in shape; however, if ΔL*, Δa*, and Δb* are equal, the solid will be spherical. As used herein, a “solid” refers to the mathematical concept of a three-dimensional figure having length, breadth, and height (or depth). An ellipsoidal volume is preferred to calculate volume because an ellipsoid generally requires the dimensional differences of ΔL*, Δa*, and Δb* to be relatively more uniform than other solids. Furthermore, it is believed that ellipsoidal volumes are more visually acceptable (i.e., less detectable color mismatch by human perception) than spherical volumes.

Ideally, the imparted colors of at least two externally visible surfaces of discrete elements will occupy a CIELab color space volume of less than about 190. The externally visible surfaces are analyzed according to the Test Method described below. Upon analysis, the inherent color of an element comprising an externally visible surface will yield L*, a*, and b* coordinates. The CIELab color space volume is then calculated utilizing the formula presented above. The resulting volume will preferably be less than about 190. More preferably, the resulting volume will be less than about 150. Even more preferably, the resulting volume will be less than about 100.

It should be recognized that the imparted colors of more than two discrete elements having an externally visible surface may occupy the aforementioned CIELab color space volumes. In calculating the color space volume for more than two elements, volume is calculated using the maximum and minimum L*, a*, and b* from a set of elements. A given set of elements will yield a set of L*, a*, and b* values. A maximum color value is selected by taking the maximum L*, the maximum a*, and the maximum b* from the set of L*, a*, and b* values. Likewise, a minimum color value is selected by taking the minimum L*, the minimum a*, and the minimum b* from the set of L*, a*, and b* values. The maximum color values and minimum color values are used to calculate V according to the formula presented above. Preferably, the imparted colors of more than two discrete elements having an externally visible surface will occupy the volume. More preferably, the imparted colors of more than three discrete elements having an externally visible surface will occupy the volume.

ii. Color Space Total Difference

One embodiment of the present invention is color matching of externally visible surfaces such that the colors have a specified CIELab color space total color difference (ΔE). In other words, this is a single number that expresses the magnitude of the difference between two colors. The value tells nothing about the nature of the color difference. Unlike the measurement of color space volume which can measure more than two colors, the total color difference measurement only can measure two colors. Characterizing color matching by the total color difference is desirable in that total color difference accounts for and considers all three dimensions within CIELab. While not being limited to this theory, such a three-dimensional measurement is believed to more fully characterize the difference in two colors. The total color difference represents the distance between two points within CIELab color space. The CIELab color space total color difference (ΔE) for a first color (L*₁, a*₁, b*₁) and a second color (L*₂, a*₂, b*₂), is calculated according to the following formula: ΔE=\sqrt{(ΔL*)²+(Δa*)²+(Δb*)²}. Within this formula, ΔL* is the difference in L* values between the two colors and is calculated by: ΔL*=L*₂− L*₁. The Δa* is the difference in a* values between the two colors and is calculated by: Δa*=a*₂− a*₁. The Δb* is the difference in b* values between the two colors and is calculated by: Δb*=b*₂− b*₁.

Ideally, at least two elements comprising an externally visible surface each comprising an imparted color will have a CIELab color space total color difference of less than about 3. The externally visible surfaces are analyzed according to the Test Method described below. Upon analysis, the inherent color of an element comprising an externally visible surface will yield L*, a*, and b* coordinates. The CIELab color space total color difference is then calculated utilizing the formula presented above. The resulting ΔE will preferably be less than about 3. Even more preferably, the resulting ΔE will be less than about 2. In one non-limiting example, the color for the overwrap 40 is a green and the color for the absorbent member 22 is green. The parameters for ΔL*, Δa*, and the Δb* are (0.35, 0.30, 0.20). The ΔE that results from this example is 0.50.

It should be recognized that imparted colors of more than two discrete elements having an externally visible surface may have the aforementioned CIELab color space total color difference. Preferably, the imparted colors of more than two discrete elements having an externally visible surface will be less than the aforementioned ΔE. More preferably, the imparted colors of more than three discrete elements having an externally visible surface will be less than the aforementioned ΔE. Comparing more than two elements implies that, no matter which two elements having an externally visible surface containing the imparted color are compared, the resulting total color difference will be less than the aforementioned ΔE values.

iii. Color Space Hue Difference

One embodiment of the present invention is color matching of externally visible surfaces such that the colors have a specified CIELab color space hue difference (ΔH). Characterizing color matching by the hue difference is desirable in that hue difference accounts for and considers all three dimensions within CIELab. While not being limited to this theory, such a three-dimensional measurement is believed to more fully characterize the difference in two colors. The hue difference represents the distance between two points within CIELab color space. The CIELab color space hue difference (ΔH) for a first color (L*₁, a*₁, b*₁) and a second color (L*₂, a*₂, b*₂), is calculated according to the following formula: ΔH=\sqrt{(ΔE)²−(ΔC)²−(ΔL*)²}. Within this formula, ΔE is the CIELab color space total color difference between the two colors and is calculated as presented above. The ΔC is the CIELab color space chroma difference between the two colors and is calculated by: ΔC=\sqrt{a*₂²+b*₁²}−\sqrt{a*₁²+b*₁²}. The ΔL* is the difference in L* values between the two colors and is calculated by: ΔL*=L*₂−L*₁

Ideally, at least two externally visible surfaces each comprising an imparted color will have a CIELab color space hue difference of less than about 4. The externally visible surfaces are analyzed according to the Test Method described below. Upon analysis, the inherent color of an element comprising an externally visible surface will yield L*, a*, and b* coordinates. Two elements are selected and the L*, a*, and b* values of the elements are inserted into the formula presented above to result in a hue difference. The resulting hue difference will preferably be less than about 4. More preferably, the resulting ΔH will be less than about 3. Most preferably, the resulting ΔH will be less than about 1.

It should be recognized that imparted colors of more than two discrete elements having an externally visible surface may occupy the aforementioned CIELab color space hue difference. Preferably, the imparted colors of more than two discrete elements having an externally visible surface will be less than the aforementioned ΔH. More preferably, the imparted colors of more than three discrete elements having an externally visible surface will be less than the aforementioned ΔH. Comparing more than two elements implies that, no matter which two elements having an externally visible surface containing the imparted color are compared, the resulting hue difference will be less than the aforementioned ΔH values.

I. Tampon of the Present Invention:

Referring again to FIG. 1, in its broadest aspect, the present invention relates to the main elements of the tampon 20: the absorbent material and the overwrap 40 independently having an L* value of greater than about 60 and a C* value of greater than about 6. If a skirt portion 44 is included, the skirt portion 44 may also have an L* value of greater than about 60 and a C* value of greater than about 6.

Referring again to FIG. 1, in another aspect, the present invention relates to color matching the imparted color of the main elements of the tampon 20: the absorbent material and the overwrap 40. If a skirt portion 44 is included, the skirt portion 44 may also be color matched to the absorbent material and/or to the overwrap 40. In other words, at least two elements are color matched by having the same color according to the Commission Internationale de l'Eclairage L*a*b* color space (hereinafter “CIELab”). The color matching can be used to provide an absorbent article holistically.

FIG. 1 shows a tampon 20 of the present invention. In general, tampon 20 comprises a compressed absorbent member 22 and a skirt portion 44 that covers absorbent member 22. Overwrap 40 may extend beyond one end of absorbent member 22 to form a skirt portion 44. A removal means, such as string 48 can be provided to facilitate removal of the tampon 20 after use. Tampons, including overwraps for use as the body contacting surface thereof, are well known in the art and need no detailed description of various alternative and optional designs.

In one embodiment, each imparted color of absorbent member 22 and the overwrap 40 can be color matched. For example, the absorbent member 22 and the overwrap 40 can be yellow. Accordingly, the L*a*b* values for this example can be (65,0,20). In yet another example, the absorbent member 22 and the overwrap 40 can be green. Accordingly, the L*a*b* values for this example can be (70,−30,0).

The absorbent member 22 and the skirt portion 44 can be color matched. For example, the absorbent member 22 and the skirt portion 44 can be red. Accordingly, the L*a*b* values for this example can be (75,20,0). In yet another example, the absorbent member 22 and the skirt portion 44 can be blue. Accordingly, the L*a*b* values for this example can be (70,−30,0).

The overwrap 40 and the skirt portion 44 can be color matched. For example, the overwrap 40 and the skirt portion 44 can be red. Accordingly, the L*a*b* values for this example can be (75,30,0). In yet another example, the overwrap 40 and the skirt portion 44 can be green. Accordingly, the L*a*b* values for this example can be (80,−30,0).

Each major element of the tampon 20 is described below.

a. Absorbent Material

The absorbent material 28 may be any suitable size and thickness suitable for compression into a tampon 20 having a vaginally insertable shape. In the embodiment shown in FIG. 2, the absorbent material 28 is generally square or rectangular, but other shapes such as trapezoidal, triangular, hemispherical, chevron and hourglass shaped are also acceptable. A typical size for absorbent material 28 prior to compression may be from about 40 mm to about 100 mm in length and from about 40 mm to about 80 mm in width. The typical range for the overall basis weight is from about 150 gsm to about 1200 gsm.

The absorbent material 28 may be a laminar structure comprised of integral or discrete layers. In other embodiments, the pad need not have a layered structure at all. The absorbent material 28 may comprise a folded structure or may be rolled. The resulting compressed absorbent member 22 of the tampon 20 may be constructed from a wide variety of liquid-absorbing materials commonly used in absorbent articles such as rayon (including tri-lobal and conventional rayon fibers), cotton, or comminuted wood pulp which is generally referred to as airfelt. Examples of other suitable absorbent materials 28 include creped cellulose wadding; meltblown polymers including coform; chemically stiffened, modified or cross-linked cellulosic fibers; synthetic fibers such as crimped polyester fibers; foam; tissue including tissue wraps and tissue laminates; or any equivalent material or combinations of materials, or mixtures of these.

Typical absorbent materials 28 comprise cotton, rayon, folded tissues, woven materials, non-woven webs, synthetic and/or natural fibers or sheeting. The tampon 20 and any component thereof may comprise a single material or a combination of materials. Additionally, superabsorbent materials, such as super polymers or absorbent gelling and open-celled foams materials may be incorporated into the tampon.

The materials for the tampon 20 can be formed into a fabric, web, or batt that is suitable for use in the absorbent material 28 by any suitable process such as airlaying, carding, wetlaying, hydroentangling, needling or other known techniques.

In another non-limiting embodiment, the absorbent material 28 and resulting compressed absorbent member 22 comprise rayon, cotton, or combinations of both materials. These materials have a proven record of suitability for use in the human body. The rayon used in the absorbent material 28 may be any suitable type typically used in disposable absorbent articles intended for in vivo use. Such acceptable types of rayon include GALAXY Rayon (a tri-lobed rayon structure) available as 6140 Rayon from Acordis Fibers Ltd., of Kelheim, Germany. SARILLE L rayon (a round fiber rayon), also available from Acordis Fibers Ltd. is also suitable. Any suitable cotton material may be used in the compressed absorbent member 22. Suitable cotton material includes, long fiber cotton, short fiber cotton, cotton linters, T-fiber cotton, card strips, and comber cotton. Preferably, the cotton layers should be scoured and bleached cotton absorbent with a glycerin finish, or other suitable finish.

If the compressed absorbent member of the present invention is layered, the layers may comprise different materials. For example, in one embodiment, the outer layers may comprise primarily rayon, while the intermediate layer or layers may comprise primarily cotton. Optionally, the entire compressed absorbent member 22 may comprise a uniform or non-uniform blend of materials throughout.

Pressures and temperatures suitable for compression are well known in the art. Typically, the absorbent material 28 and the overwrap 40 are compressed in the radial direction and optionally axially by any means well known in the art. While a variety of techniques are known and acceptable for these purposes, a modified tampon compressor machine available from Hauni Machines, Richmond, Va., is suitable.

b. Overwrap:

Referring to FIG. 2, in the embodiments shown, the overwrap material 40 is generally rectangular, but other shapes such as trapezoidal, triangular, hemispherical, chevron, hourglass shaped, “T” and “L” shaped are also acceptable. Optimally, the overwrap 40 may correspond to the shape of the absorbent material 28. The overwrap 40 is positioned around the absorbent material 28 so that the overwrap 40 may be proximate with the insertion end 38 of the absorbent material 28. In this regard, the overwrap 40 could exactly match up to the insertion end 38 or could for example extend from about 2 mm to about 8 mm over the insertion end 38. As well, the overwrap 40 may extend beyond the withdrawal end 42 to form a skirt portion 44 as discussed below.

Because the overwrap 40 can be wrapped in the various configurations, the width of the overwrap 40 may vary. The width of the overwrap 40 may be wider or less wide than the measure of the longitudinal or transverse axis of the absorbent material 28 it is being wrapped around.

The overwrap 40 substantially covers both the first surface and the second surface of the absorbent material 28. “Substantially covers” in this case means that the overwrap 40 covers at least about 75%, optionally at least about 90% of the combined surface area of the first surface and the second surface. Thus, for example, the overwrap 40 “substantially covers” the first surface and the second surface of the absorbent material 28 when it covers 100% of the first surface and 50% of the second surface. The overwrap 40 may be wrapped around the longitudinal axis “L” or the transverse axis “T” as shown in the attached figures in another embodiment. As well, two or more separate pieces of overwrap 40 can sandwich the absorbent material 28.

The overwrap 40 may be joined to the absorbent material 28 by any variety of means. The overwrap 40 may be joined to itself or to the absorbent material 28. For example, one portion of overwrap 40 may be joined to an opposed portion of the overwrap 40 or the absorbent member 22 using any suitable adhesive or heat/pressure bonding means. Such adhesive may extend continuously along the length of attachment or it may be applied in a “dotted” fashion at discrete intervals. One method of heat bonding includes thermally bonding, fusion bonding, or any other suitable means known in the art for joining such materials. Alternatively, the overwrap 40 may be joined to the absorbent material 28 along with the withdrawal cord 48 by stitching as shown in FIG. 2. Such stitching may use natural or synthetic thread.

c. Skirt Portion

Referring to FIG. 2, the overwrap 40 may extend beyond the withdrawal end 42 to form a skirt portion 44. The length of the skirt portion 44 is not critical. Typically, the overwrap 40 can extend from about 2 mm to about 30 mm beyond the withdrawal end 42 of the absorbent material 28. Typically, the overwrap 40 extends from about 5 mm to about 20 mm beyond the withdrawal end 42 of the absorbent material 28. In one embodiment, the skirt portion 44 may not be compressed.

Referring to FIG. 1, both the compressed absorbent member 22 and skirt portion 44 of the overwrap 40 may reside entirely within the vaginal cavity of the wearer during use of the tampon 20. This is achieved by the relative closeness of the skirt portion 44 to the withdrawal end 42 of the absorbent material 28 as well of the relative size compared to the overall size of the tampon 20. In particular embodiments, only the withdrawal cord 48 or other withdrawal means resides externally to the orifice of the vagina.

d. Optional Components

Referring to FIG. 1, optionally, the tampon of the present invention could include an additional overwrap 40 that is non-aggressive. This additional overwrap 40 would substantially cover the overwrap 40 that substantially covers the exterior surface 26 of the compressed absorbent member 22 of the tampon 20. The additional overwrap 40 need only extend as to be proximate with the withdrawal end 42 of the absorbent material 28 so that the entire skirt portion 44 of the tampon 20 is left uncovered by the additional overwrap 40. This additional overwrap 40 could be added prior to or subsequent to compression.

In one embodiment, the tampon 20 of the present invention may comprise a withdrawal means 48. The withdrawal means 48 could be joined to the tampon 20 and graspable for digital removal after use. The withdrawal means 48 may be joined to at least the primary compressed absorbent member 22 and extends beyond at least the withdrawal end 42. The withdrawal means 48 may be joined in any suitable manner known in the art including sewing, adhesive attachment, or a combination of known bonding methods. The withdrawal means 48 may be joined to any suitable location on the tampon.

Any of the withdrawal means 48 currently known in the art may be used as a suitable withdrawal mechanism. In addition, the withdrawal means 48 can take on other forms such as a ribbon, loop, tab, or the like. The withdrawal means 48 may be integral with the absorbent material 28.

The tampon 20 of the present invention may be inserted digitally or through the use of an applicator. Any of the currently available tampon applicators may also be used for insertion of the tampon of the present invention. Such applicators are of typically a “tube and plunger” type arrangement and may be plastic, paper, or other suitable material. Additionally, a “compact” type applicator is also suitable.

II. Process of Making:

While several methods of making the tampon of the present invention should be apparent to one of skill in the art in light of the disclosure herein, methods of making a tampon of the present invention can be found in Ser. No. 10/430,916, filed May 7, 2004, entitled “Tampon With Joined Skirt Portion”, to “Karapasha, et al.” and Ser. No. 09/993,988, filed Nov. 16, 2001, entitled “Tampon With Fluid Wicking Overwrap with Skirt”, to “Hasse, et al.”

Test Method

Externally visible surfaces are tested in a dry state and at an ambient humidity of approximately 50%±2%. Reflectance color is measured using the Hunter Lab LabScan XE reflectance spectrophotometer obtained from Hunter Associates Laboratory of Reston, Va. The spectrophotometer is set to the CIELab color scale and with a D50 illumination. The Observer is set at 10° and the Mode is set at 45/0°. Area View is set to 0.125″ and Port Size is set to 0.20″ for films; Area View is set to 1.00″ and Port Size is set to 1.20″ for nonwovens and other materials. The spectrophotometer is calibrated prior to sample analysis utilizing the black and white reference tiles supplied from the vendor with the instrument. Calibration is done according to the manufacturer's instructions as set forth in LabScan XE User's Manual, Manual Version 1.1, August 2001, A60-1010-862. If cleaning is required of the reference tiles or samples, only tissues that do not contain embossing, lotion, or brighteners should be used (e.g., Puffs® tissue). Any sample point on the externally visible surface of the element containing the imparted color to be analyzed should be selected. Ideally, sample points are selected so as to be close in perceived color. A single ply of the element is placed over the spectrophotometer's sample port. A single ply, as used within the test method, means that the externally visible surface of the element is not folded. Thus, a single ply of an externally visible surface may include the sampling of a laminate, which itself is comprised of more than one lamina. The sample point comprising the color to be analyzed must be larger than the sample port to ensure accurate measurements. A white tile, as supplied by the manufacturer, is placed behind the externally visible surface. The L*, a*, and b* values are read and recorded. The externally visible surface is removed and repositioned so that a minimum of six readings are obtained for the externally visible surface. If possible (e.g., the size of the imparted color on the element in question does not limit the ability to have six discretely different, non-overlapping sample points), each of the readings is to be performed at a substantially different region on the externally visible surface so that no two sample points overlap. If the size of the imparted color region requires overlapping of sample points, only six samples should be taken with the sample points selected to minimize overlap between any two sample points. The readings are averaged to yield the reported L*, a*, and b* values for a specified color on an externally visible surface of an element.

In calculating the color space volume, V, maximum and minimum L*, a*, and b* values are determined for a particular set of elements to be color matched. The maximum and minimum L*, a*, and b* values are used to calculate V according to the formula presented above.

### EXAMPLES

The following is a listing of examples illustrating various embodiments of the present invention. It would be obvious to those skilled in the art that various other changes and modifications can be made without departing from the spirit and scope of the invention.

Examples 1-2, provided below, are tampons of the present invention. A tampon 20 is to be tested having the same general construction as the TAMPAX® Pearl Plastic tampon. Suitable tampon construction is detailed in U.S. Pat. No. 6,258,075. The tampon of Example 1 and 2 are substantially the same as those commercially available except for the absorbent member and the overwrap. The absorbent member is 100% rayon. The absorbent member is colored by dyeing. The overwrap is a starch bonded overwrap which is 100% rayon and 15 gsm. The overwrap is colored by dyeing.

### Example 1

The absorbent member, the overwrap, and the skirt of the example are tested according to the test method described above. The three elements tested (the absorbent member and the overwrap) fall within a color space volume of 97 according to the calculation described above.

Table 1 provides that three elements (the absorbent member, the overwrap, and the skirt) have a maximum ΔE* of 2.2. Calculation of ΔE* is performed on an element-to-element basis as described above. Given the three elements, three comparisons may be performed: the absorbent member, the overwrap, and the skirt. The ΔE* values for the three comparisons are 2.0, 2.1, and 2.2, respectively. The “-” in the Table indicates that element to element is not analyzed. In light of these values, the three elements are color matched in that all of the comparisons result in total color differences of less than about 2.2.

TABLE 1
ΔE*

| | Absorbent Member | Overwrap | Skirt |
|---|---|---|---|
| Absorbent Member | — | 2.0 | 2.1 |
| Overwrap | 2.0 | — | 2.2 |
| Skirt | 2.1 | 2.2 | — |

Table 2 provides that two elements (absorbent member and overwrap) have a maximum ΔH* of 0.9. Calculation of ΔH* is performed on an element-to-element basis as described above. Given the two elements, two comparisons may be performed: absorbent member and the overwrap. The ΔH* values for the two comparisons are 0.6 and 0.9, respectively. The “-” in the Table indicates that element to element is not analyzed. In light of these values, the two elements are color matched in that all of the comparisons result in hue difference of less than 0.9.

TABLE 2
ΔH*

| | Absorbent Member | Overwrap |
|---|---|---|
| Absorbent Member | — | 0.9 |
| Overwrap | 0.6 | — |

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Detailed Description are in relevant part incorporated herein by reference; the citation of any document is not to be construed as an admission that it is prior art with respect to the present invention.

What is claimed is:

1. A tampon comprising:
an externally visible surface comprising an imparted color, wherein said imparted color has an L* value of greater than about 60 and a C* value of greater than about 6.

2. A tampon comprising:
an absorbent material having a first surface opposed to a second surface, wherein said absorbent material comprises an externally visible surface comprising an imparted color,
wherein said imparted color has an L* value of greater than about 60 and a C* value of greater than about 6.

3. A tampon comprising:
an absorbent material having a first surface opposed to the second surface, and an insertion end opposed to a withdrawal end and
an overwrap covering at least a portion of said first surface and at least a portion of said second surface of said absorbent material comprising an imparted color, wherein said imparted color of said absorbent material and said imparted color of said overwrap independently have an L* value of greater than about 60 and a C* value of greater than about 6.

4. A tampon according to claim 3 wherein said overwrap extends beyond the withdrawal end of said first surface of said absorbent material to define a first skirt portion comprising an imparted color and
said overwrap extends beyond the withdrawal end of said second surface of said absorbent material to define a second skirt portion comprising an imparted color, said imparted color of said skirt portion comprising an L* value of greater than about 60 and a C* value of greater than about 6.

5. A tampon according to claim 4 wherein said first skirt portion is joined to said second skirt portion.

6. The tampon of claim 3 wherein the difference ΔL* between said absorbent material and said overwrap is less than about 3.

7. The tampon of claim 3 wherein the difference in color value ΔE* between said absorbent material and said overwrap is less than about 3.

8. The tampon of claim 3 wherein said absorbent material and said overwrap have the same color.
